(19) United States
(12) Patent Application Publication
(10) Pub. No.: US 2010/0275677 A1
(43) Pub. Date: Nov. 4, 2010

(54) DETECTING FLUID IN A CONDUIT ARRANGEMENT

(76) Inventor: Dennis Charles Parkins Gibbs, Cheltenham (GB)

Correspondence Address: KING & SCHICKLI, PLLC, 247 NORTH BROADWAY, LEXINGTON, KY, 40507, US

(21) Appl. No.: 12/732,507

(22) Filed: Mar. 26, 2010

Related U.S. Application Data
(60) Provisional application No. 61/174,120, filed on Apr. 30, 2009.

(51) Int. Cl. G01N 33/00

(52) U.S. Cl. 73/53.01

(57) ABSTRACT

Apparatus adapted to detect fluid in a conduit arrangement (100, 101) includes a first fluid sensor (102) and a second fluid sensor (104). A fluid detection device (105) is in communication with the first and the second sensors, the device being configured to output a fluid detection signal when both the first and the second sensors detect presence of fluid. In use, the first and the second sensors are positioned at different positions along a conduit arrangement and/or the first and the second sensors are disposed at different radial angles with respect to an axis of a conduit arrangement.

The present application claims priority from U.S. Provisional Patent Application Ser. No. 61/174,120 filed on Apr. 30, 2009.

### FIELD OF THE INVENTION

The present invention relates to detecting fluid in a conduit arrangement.

### BACKGROUND TO THE INVENTION

Many different types of fluid detectors are available. Some of these are simple in design and comprise a sensing element that is located within a conduit where fluid is to be detected and produces an output when it comes into contact with fluid. In some cases such detectors are insufficient because the presence of residual fluid can result in false readings, i.e. an indication that there is fluid present when there is only a small/insufficient amount. For example, in a shower pump if a sufficient amount of fluid does not pass through the pump chamber then the fins of the rotor can come into contact with the internal wall of the pump chamber, which leads to premature wear due to friction. There are many other instances where it is desirable to detect whether fluid is present in, or flowing through, a conduit, or whether there is a minimum quantity of fluid present.

### SUMMARY OF THE INVENTION

Embodiments of the present invention are intended to detect fluid, particularly in a conduit arrangement.

According to one aspect of the present invention there is provided apparatus adapted to detect fluid in a conduit arrangement, the apparatus including:

a first fluid sensor;

a second fluid sensor, and

a fluid detection device in communication with the first and second sensors, the device being configured to output a fluid detection signal when both the first and the second sensors detect the presence of fluid,

where, in use, the first and second sensors are positioned at different positions along a main direction of a conduit arrangement and/or the first and the second sensors are disposed at different radial angles with respect to an axis of the conduit arrangement.

In some embodiments the first sensor may comprise at least a portion of the conduit arrangement, e.g. an electrically-conductive/metallic portion of the conduit arrangement that can act as an electrical terminal for a device.

In other embodiments, the first and second sensors are positioned spaced-apart at different locations along a length of a conduit arrangement. The first sensor can have a height, with respect to a base of the conduit arrangement, which is different to a corresponding height of the second sensor.

The relative heights/radial angles of the first and second sensors may be arranged such that fluid flowing through the conduit arrangement (or a minimum quantity of fluid) is detected by the sensors, but residual fluid is not detected (as residual/standing fluid will not reach/contact the sensors). In some embodiments, each of the first and second sensors includes a plurality of sensing devices. For example, each of the first and second sensor may include a pair of sensing devices, a first said sensing device in each pair being positioned so as to oppose the other sensing device in the pair (e.g. positioned at 180° relative to each other). In some embodiments, the opposed sensing devices of the first sensor are arranged at radial angles of around 90° relative to opposed sensing devices of the second sensor. The opposed sensing devices of the first sensor may be arranged at radial angles different to those of the opposed sensing devices of the second sensor.

The first and/or the second sensor may output a sensor fluid detection signal to the device when all (or more than one) of its sensing devices detect the presence of fluid. The first and/or the second sensor may include a delay device that delays the output of the sensor fluid detection signal to the device.

The fluid detection signal of the device may activate/de-active a function. For example, the function may comprise activating/de-activation a pump that drives fluid through the conduit arrangement. The device may further include an input device, e.g. a switch, for allowing manual activation/de-activation of the function. The first sensor may be located at or adjacent an inlet of a component, e.g. a pump, that is in flow communication with the conduit arrangement, and the second sensor may be located at or adjacent an outlet of the component.

If the conduit arrangement is electrically non-conductive then the first and second sensor may be formed of conductive material and be located in or on a surface of the conduit arrangement. If the conduit arrangement is electrically conductive then the first and/or second sensor may each include a conductive member isolated from the conduit arrangement. For example, the first and/or second sensor may comprise a metal member surrounded by an insulator, e.g. a rubber ring. The sensors may comprise a resistive, capacitative, Hall Effect, or inductive sensor.

The device may further include a formation that, in use, increases a surface area of the conduit arrangement between the first sensor and the second sensor.

According to another aspect of the present invention there is provided a fluid conduit arrangement including a fluid detector, the arrangement including:

first sensor and second sensors, wherein the first and second sensors are positioned at different locations along a main direction of the conduit arrangement along which fluid can flow, and/or the first and second sensors being disposed at different radial angles with respect to an axis of the conduit arrangement.

The conduit arrangement may comprise a pipe or the like having a circular cross-section. In such cases, the first sensor may be disposed at a different radial angle to the second sensor, thereby giving the first and second sensor different heights with respect to a base of the conduit arrangement (when in a horizontal orientation).

The conduit arrangement may include at least one formation that increases its internal surface between the first sensor and the second sensor. The formation may include a ring-shaped protrusion on an inner surface of the conduit. The ring-shaped protrusion may include a tapering inner surface.

According to another aspect of the present invention there is provided at least one conduit including Apparatus substantially as described herein.

According to another aspect of the present invention there is provided a pump arrangement including a conduit and at least one fluid detection device substantially as described herein.

According to a further aspect of the present invention there is provided a apparatus adapted to detect fluid in a conduit arrangement, the apparatus including:

a fluid sensor including a plurality of sensing devices, and

a fluid detection device in communication with the sensor, the device being configured to output a fluid detection signal when all or some of the sensing devices detect the presence of fluid,

where, in use, a height of at least one of the sensing devices, with respect to a base of the conduit arrangement, differs from a height of the other sensing device(s).

The device may further include a second fluid sensor that, in use, is positioned spaced-apart at a different location along a length of a conduit arrangement to the first sensor, the device being configured to output a fluid detection signal when both the first and the second sensors detect the presence of fluid.

The second sensing device may include a plurality of sensing devices and at least one of the sensing devices of the second sensor may have a height that differs from a height of the other sensing devices of the second sensor and/or differs from a height of at least one sensing device of the first sensor.

The heights of the sensing devices of the first and/or the second sensor may be arranged so that fluid flowing through the conduit arrangement is detected by the sensing devices, but residual fluid is not detected, as it will not reach/contact the sensing devices.

According to another aspect of the present invention there is provided a method of detecting fluid in a conduit arrangement, the method including fitting (and operating) Apparatus substantially as described herein to a conduit arrangement.

Whilst the invention has been described above, it extends to any inventive combination of features set out above or in the following description. Although illustrative embodiments of the invention are described in detail herein with reference to the accompanying drawings, it is to be understood that the invention is not limited to these precise embodiments. As such, many modifications and variations will be apparent to practitioners skilled in the art. Furthermore, it is contemplated that a particular feature described either individually or as part of an embodiment can be combined with other individually described features, or parts of other embodiments, even if the other features and embodiments make no mention of the particular feature. Thus, the invention extends to such specific combinations not already described.

### BRIEF DESCRIPTION OF THE DRAWINGS

The invention may be performed in various ways, and, by way of example only, embodiments thereof will now be described, reference being made to the accompanying drawings in which:

FIG. 1A is a schematic sectional view through a pipe including an example of the fluid detector;

FIGS. 1B and 1C are cross sectional views through lines B-B′ and C-C′, respectively, of FIG. 1;

FIG. 2 is a block diagram of a circuit of the detector;

FIG. 3 is a diagram of the example circuit;

FIG. 4 is a schematic side view of a conduit including another example of the fluid detector;

FIG. 5A is a schematic side view of a conduit including yet another example of the fluid detector, and

FIG. 5B is a semi-transparent end view of the conduit and detector of FIG. 5A.

### DETAILED DESCRIPTION OF THE DRAWINGS

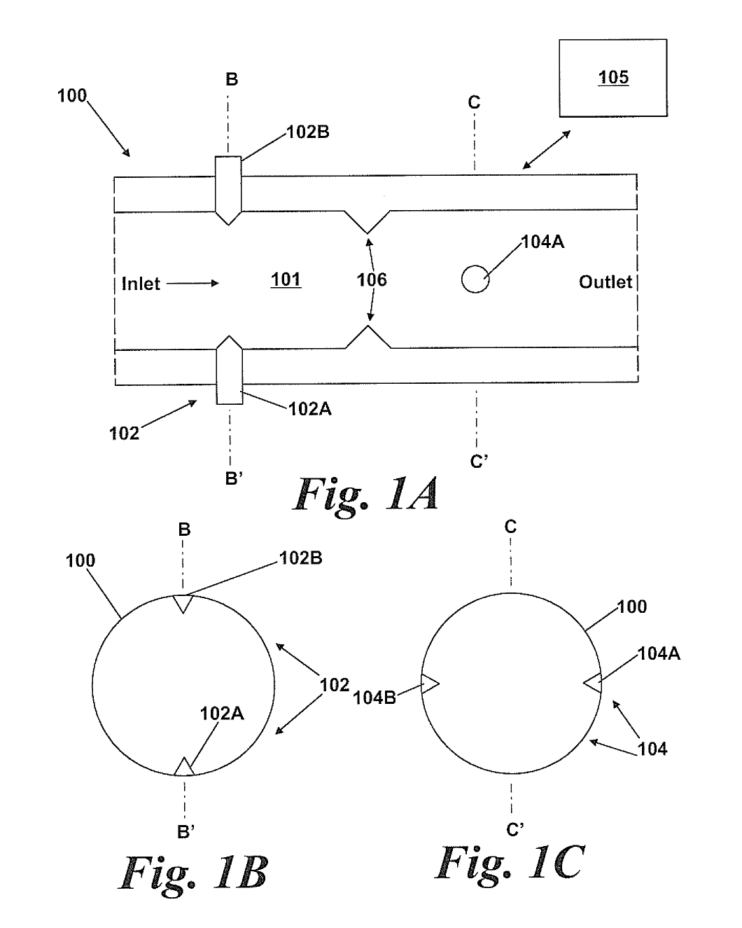

Referring first to FIGS. 1A-1C, a pipe 100 defines a conduit 101 through which fluid is intended to pass, e.g. in the direction indicated by the arrow. In the example the pipe 100 is a section of a tubular pipe that may be formed of electrically non-conductive material, e.g. plastic or any other suitable substance. Although the pipe shown in the Figures is straight and horizontal, it will be understood that the fluid detector described herein can be arranged to detect fluid in conduit arrangements that are oriented in a vertical (or other) direction and may not be straight. It will also be understood that types of conduit arrangements other than pipes can benefit from the detector, e.g. partially open channels having straight walls. Further, the conduit arrangements in which the detector is fitted may be formed of a single piece or may comprise several different components fitted together and the term “conduit arrangement” is intended to be broadly interpreted and covers various different arrangements that can vary in construction, design and dimensions from the illustrated example. Further, the conduit arrangement can include items, e.g. containers/boxes, positioned along the length of the conduit and in flow communication with the conduit.

The pipe 100 includes a first sensor 102 comprising a pair of metallic probes 102A, 102B. In the example the two probes comprise metallic elements fitted in the plastic pipe, with one end of each probe projecting through the inner surface of the pipe. It will be appreciated the probes 102 are only one example of a type of sensor that can be used; resistive, capacititive or inductive sensors may be used and may be fitted in, on (externally) or adjacent to the conduit, according to the capabilities of the particular sensors being used. Thus, in other embodiments a sensor may comprise more than two individual sensing devices, and in other examples may only comprise one sensing device.

The first sensing device 102A of the illustrated example is located in the lowermost part of the pipe (when it is in a horizontal orientation). The second sensing device 102B is located in the uppermost part of the pipe and opposes the first device 102A, i.e. it is at an angle of 180° with respect to a central axis of the conduit compared with the radial angle of the first sensing device 102A. It will be appreciated that having the two devices at angles of 180° with respect to each other is exemplary only and the sensing devices may be positioned at other angles on the circumference of the pipe 100. However, it is advantageous that one of the probes is located in a portion of the conduit where residual fluid will not normally be present. That is, the upper sensing device 102B will normally only come into contact with fluid when the pipe is substantially full of fluid and/or when the fluid is flowing at the expected rate through the pipe.

In the illustrated arrangement the two separate sensing devices 102A, 102B that are connected together in an electrical circuit and transfer outputs to a circuit 105 when they both detect the presence of fluid. Thus, the two individual sensing devices can be thought of as forming a first sensor 102 that detects the presence of flowing fluid at the points in the conduit where the sensing devices are located. It will be understood that the individual sensing devices that act together as a single sensor need not be located exactly in the same position along the length of the conduit and variations are possible, e.g. they may be located a certain distance apart.

In the example of FIG. 1 there is a second sensor 104 that also comprises a pair of sensing devices 104A, 104B. However, again, the second sensor 104 can comprise one or more individual sensing devices and these may be arranged in any manner, as discussed above in relation to the first sensor 102. In the illustrated example the second pair of sensing devices 104A, 104B are arranged on the circumference of the pipe 100 at angles of around 90° relative to the first pair of sensing devices. However, again, it will be understood that the exact location of the second sensing device relative to the first one may be varied. For example, in another embodiment, the sensing devices of the first or second sensors can have a greater height and/or surface area than the sensing devices of the other sensor and so detect the presence of fluid at different portions of the conduit. Further, the distance between the first 102 and second 104 sensors may vary.

The pipe 100 further includes a formation 106 located between the first and second sensors 102, 104. In the example the formation comprises a ring 106 having a tapered inner surface; however, it will be appreciated that this is only one example of a formation that will increase the surface area of the conduit between the first and second sensors in order to assist with avoiding having residual fluid present adjacent the sensors. More than one such formation may be provided between a pair of adjacent sensors. Further, more than two sensors 102, 104 may be arranged along the length of the conduit arrangement.

The sensors can be in communication with (e.g. by means of insulated wires or wireless communications devices) a device, such as circuit 105 that will be described in more detail below. In embodiments where the conduit is formed of an electrically conductive material the individual sensing devices may be isolated from the conduit material, e.g. by means of a rubber casing. In yet another variation, where the conduit arrangement is at least partially formed of an electrically conductive material (e.g. copper piping) then one of the sensors/sensing devices may be formed by that conductive material, e.g. act as a negative electrical terminal. In such embodiments, the second (and possibly further) sensor can be formed of at least one sensing device that is isolated from the conductive material of the conduit arrangement. Thus, the apparatus comprising the sensors 102, 104 and the circuit 105 may be considered to constitute a sensing unit that can be used to detect fluid. In some cases there may be more than one such sensing unit connected to different positions of a conduit arrangement or other location where a fluid is to be detected.

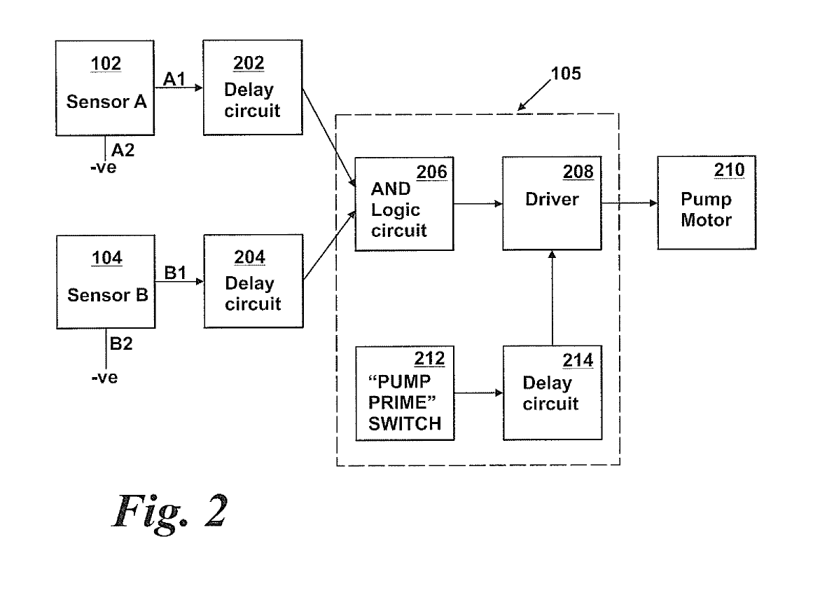

Turning to FIG. 2, a block diagram of a circuit 105 that is intended for use in a shower pump is shown. In general, the circuit will perform a predetermined function when it receives signals from all (or some, depending on the particular configuration) sensors indicating that they detect fluid in the conduit arrangement. Different sensors (or combinations of sensors) can activate different functions, e.g. alarms. In the case of a shower pump, the function can include switching off the pump motor when fluid is not detected and switching it back on when fluid is detected by the sensors. In the example of a shower pump a first fluid sensor may be located adjacent an inlet of the pump chamber and a second sensor may be located adjacent to the outlet of the pump chamber.

In the example of FIG. 2, each of the sensors 102, 104 is connected to the circuit 105 via a respective delay circuit 202, 204. The delay circuit can be beneficial in the event of a temporary interruption to the flow of fluid through the conduit so that short/non-harmful interruptions in flow do not necessarily trigger the circuit function. The two delay circuits 202, 204 transmit their outputs to a logical AND circuit 206 in the circuit 105, which controls a driver 208 for the pump motor 210. It can also be beneficial to provide a “pump prime” switch 212 for manually resetting the pump if a failure is detected. The switch 212 is connected via a delay circuit 214 to the driver 208. In other applications further user input may be available, e.g. override the detector function or provide additional functionality.

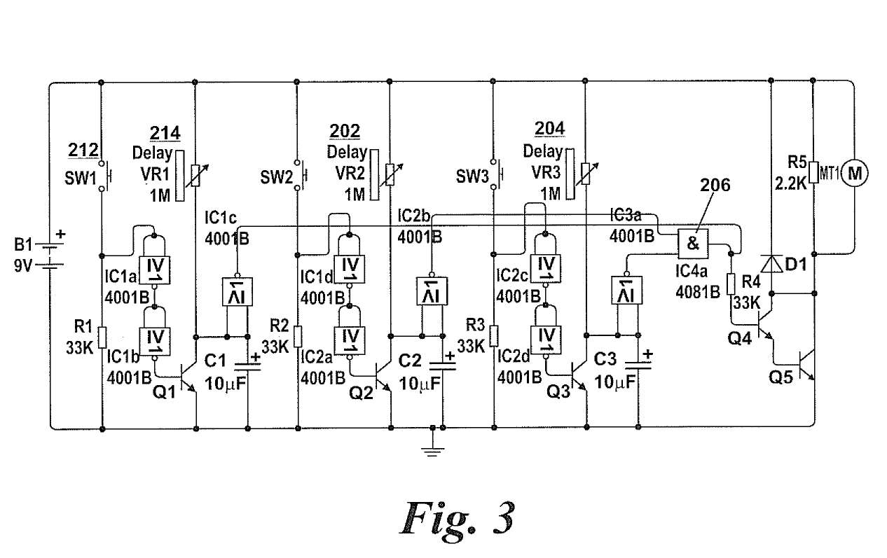

FIG. 3 is a detailed circuit diagram showing an example of how the arrangement outlined in FIG. 2 may be implemented. It will be understood that the illustrated circuit is only one example of a device that can detect fluid based on the sensor units' outputs and that other software and/or hardware-based implementations are possible. For instance, the sensor units may be connected to one or more computing devices configured to execute code that can process the sensor outputs in various ways. For example, the computer may issue a (local and/or remote) warning signal, or control a piece of hardware in accordance with the result of processing the signals.

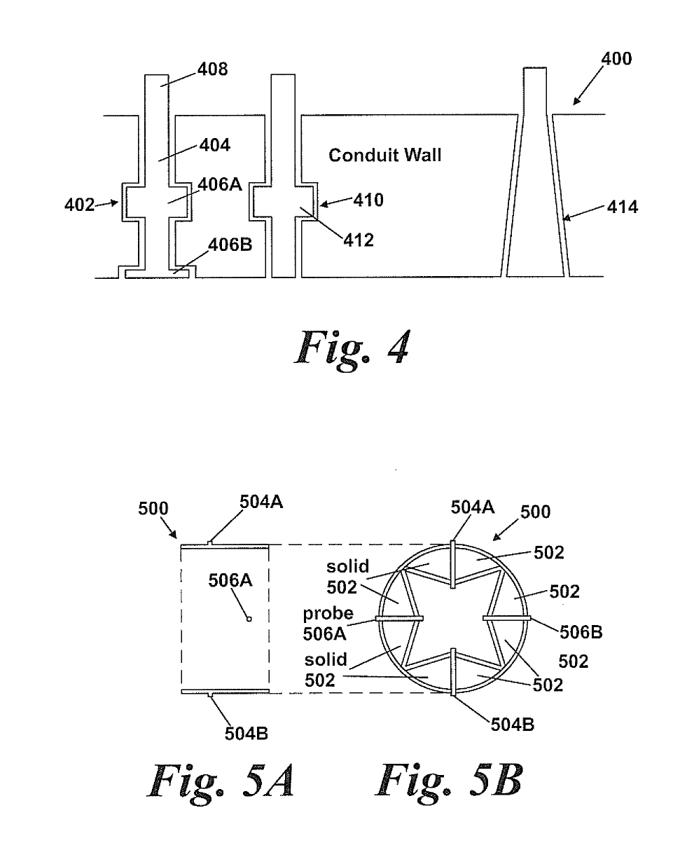

Referring to FIG. 4, other examples of probes that function as sensor units that may be used in a conduit arrangement 400 are shown. These example probes are intended to form part of the wall/surface of the conduit and lie substantially flush with its inner surface (as opposed to the probes in FIG. 1, which protrude into the conduit space). Having flush probes can offer the advantage of avoiding collecting debris. This can be useful because embodiments of the fluid detector can operate in a variety of harsh environments.

The first example probe 402 in FIG. 4 is formed to be inserted into part of the conduit wall. For ease of understanding gaps are shown between the probes of the Figure and adjacent portions of the conduit wall, but it will be appreciated that in practice the probes will be sealed to the conduit wall. The probes can be fitted in the wall by cutting out a portion of the conduit wall (either during of after manufacture), or the conduit may be formed with integrated probes. The first probe 402 includes a main elongate portion 404 including a first projection 406A part-way up the height of the conduit wall and another projection 406B adjacent the base of the wall. The probes may extend around the outer wall of the conduit, or only along part of the wall. An upper portion 408 of the probe extends upwards from the top of the conduit wall and can act as a terminal for connection to a circuit/computer.

The second example probe 410 is similar to the first one 402, except that it only includes one transverse projection 412. The third example probe 414 includes a tapering portion having a wider base adjacent a lower portion of the conduit wall. Having shapes such as these increases the surface area of parts of the probe, which provides a larger electrode for detection of fluid. It will be appreciated that the shapes shown are exemplary only and many variations are possible.

FIGS. 5A and 5B show a further example of a fluid detector. In this version, the conduit arrangement 500 (portion only shown) includes an irregular inner surface 502. There may be a space between the surface and the outer wall of the conduit, but it is preferred that it is solid. A first probe pair 504A, 504B is located at a first location along the length of the conduit and a second probe pair 506A, 506B is located at a different location. One probe 504A of the first pair extends through the outer surface of the conduit and into the inner space of the conduit, at an upper location as shown in the Figures. The other probe 504B of the first pair extends in a similar manner through a (diametrically-opposed) lower portion of the conduit. One probe 506A of the second pair extends through the outer surface of the conduit and into the inner space of the conduit, at the left-hand side around half-way up the height of the conduit as shown in the Figures. The other probe 506B of the second pair extends in a similar manner through a diametrically-opposed side portion of the conduit. The inner surface 502 of the conduit arrangement may be formed integrally during manufacture, or may be a separate insert that is fixed to at least part of the length of the conduit (either during or after manufacture). Having the probes firmly fixed to the conduit makes the detector more resilient and means that they will not normally be displaced by internal pressures within the conduit. Having the probes located between the V-shaped cavities also helps improve fluid detection when the conduit is not in horizontal orientation.

It will be appreciated that the pump application described above is only one instance of where the fluid detectors can be used. In general, the fluid detector can be arranged to operate at any orientation, including where fluid is pushed in an upward direction. The detector may be used in a variety of domestic or industrial installations, e.g. central heating systems, nuclear power stations, etc. Another specific example of a suitable application is the bilge pump onboard a ship, which is operated by a bilge pump switch. At sea the ship may heel over due to tides, winds, etc, and so the physical orientation of the pump can change. However, the seawater within the pump follows the force of gravity, which makes the water level generally equal to that of the sea level. If there is not enough water within the pump then it can seize up and so the fluid detector described herein can be used to switch off the pump when lack of sufficient water is detected. Another specific example includes a water tap/fawcett in a caravan or mobile home, which may not be located on level ground. When such a tap is turned on, the water the pump is used to draw water from a tank it is desirable to use a fluid detection system to check for the presence of water. Yet another specific example is an industrial liquid supply system, e.g. milk delivery tanker, where it can be desirable to know when fluid is present in a particular part of the system.

The detector can also be used in flood warning/rainwater detection applications. For instance, a funnel can be used to collect rainwater that has passed through a conduit with the sensor units. The conduit may run down natural slopes. The output of the sensors can be transmitted by a wireless network to a fire station or the like that can view the water detected on an electronic map. The height of rainwater may be measure and/or the time interval between when it reaches various of the sensor units in order to give a picture of the flood risk. The sensors may be powered by solar cells.

I claim:

1. Apparatus adapted to detect fluid in a conduit arrangement, the apparatus including:
a first fluid sensor;
a second fluid sensor, and
a fluid detection device in communication with the first and the second sensors, the device being configured to output a fluid detection signal when both the first and the second sensors detect presence of fluid,
where, in use, the first and the second sensors are positioned at different positions along a conduit arrangement and/or the first and the second sensors are disposed at different radial angles with respect to an axis of a conduit arrangement.

2. Apparatus according to claim 1, wherein the first sensor comprises at least a portion of the conduit arrangement, e.g. a metallic portion of the conduit arrangement.

3. Apparatus according to claim 1, wherein the first and the second sensors are positioned spaced-apart at different locations along a length of a conduit arrangement.

4. Apparatus according to claim 1, wherein the first sensor has a height, with respect to a base of the conduit arrangement, which is different to a corresponding height of the second sensor.

5. Apparatus according to claim 1, wherein the relative radial angles of the first and the second sensors are arranged such that fluid flowing through the conduit arrangement (or a minimum quantity of fluid) is detected by the sensors, but residual fluid is not detected.

6. Apparatus according to claim 1, wherein each of the first and the second sensors includes a plurality of sensing devices.

7. Apparatus according to claim 6, wherein each of the first and the second sensor includes a pair of sensing devices, a first said sensing device in each pair being positioned so as to oppose the other sensing device in the pair.

8. Apparatus according to claim 7, wherein the sensing devices in the pair are positioned at 180° relative to each other.

9. Apparatus according to claim 7, wherein the opposed sensing devices of the first sensor are arranged at radial angles different to those of the opposed sensing devices of the second sensor.

10. Apparatus according to claim 6, wherein the first and/or the second sensor outputs a sensor fluid detection signal to the device when all (or more than one) of its said plurality of sensing devices detect fluid.

11. Apparatus according to claim 1, wherein the conduit arrangement is electrically non-conductive and the first and the second sensor are formed of conductive material and are located in or on a surface of the conduit arrangement.

12. Apparatus according to claim 1, wherein the conduit arrangement is electrically conductive and the first and/or the second sensor each include a conductive member isolated from the conduit arrangement.

13. Apparatus according to claim 12, wherein the first and/or the second sensor comprise a metal member surrounded by an insulator, e.g. a rubber ring.

14. Apparatus according to claim 1, further including a formation that, in use, increases a surface area of the conduit arrangement between the first sensor and the second sensor.

15. Apparatus according to claim 1, wherein at least one of the first and second sensors is flush with an internal wall of the conduit arrangement and does not protrude into space within the conduit arrangement.

16. Apparatus according to claim 1, wherein the fluid detection signal of the device activates/de-actives a function.

17. Apparatus according to claim 16, further including an input device for allowing manual activation/de-activation of the function.

18. Apparatus adapted to detect fluid in a conduit arrangement, the apparatus including:
a first fluid sensor;
a second fluid sensor, and
a fluid detection device in communication with the first and the second sensors, the device being configured to output a fluid detection signal when both the first and the second sensors detect presence of fluid,
where, in use, the first and the second sensors are disposed at different radial angles with respect to an axis of a conduit arrangement, and
wherein each of the first and the second sensors includes a pair of sensing devices and the sensing devices of the first sensor are arranged at radial angles different to those of the sensing devices of the second sensor.

19. A fluid conduit arrangement including apparatus according to claim 1.

20. A method of detecting fluid in a conduit arrangement, the method including fitting apparatus according to claim 1 to a conduit arrangement.
